(19) United States
(12) Patent Application Publication
(10) Pub. No.: US 2014/0259233 A1
(43) Pub. Date: Sep. 11, 2014

(54) CADMIUM ABSORPTION REGULATORY GENE, PROTEIN AND CADMIUM ABSORPTION-INHIBITING RICE PLANT

(71) Applicant: NATIONAL INSTITUTE FOR AGRO-ENVIRONMENTAL SCIENCES, Ibaraki (JP)

(72) Inventors: Satoru Ishikawa, Ibaraki (JP); Masato Kuramata, Ibaraki (JP); Tadashi Abe, Ibaraki (JP); Masato Igura, Ibaraki (JP); Hiromi Nakanishi, Tokyo (JP); Naoko Nishizawa, Tokyo (JP)

(73) Assignee: NATIONAL INSTITUTE FOR AGRO-ENVIRONMENTAL SCIENCES, Ibaraki (JP)

(21) Appl. No.: 14/356,284

(22) PCT Filed: Oct. 23, 2012
(86) PCT No.: PCT/JP2012/077300

§ 371 Date: May 5, 2014

(30) Foreign Application Priority Data
Nov. 4, 2011 (JP) 2011-242041

(51) Int. Cl. C12N 15/82

(52) U.S. Cl. CPC C12N 15/8243
USPC 800/320.2; 536/23.5; 530/350; 435/320.1; 435/419

(57) ABSTRACT

Provided are a transporter gene involved in the promotion or inhibition of Cd absorption by roots, a mutant gene thereof and a transporter protein thereof, as well as a rice plant in which Cd absorption is inhibited and a method for selecting and raising a Cd absorption-inhibiting rice plant, which are a gene encoding a transporter protein involved in the regulation of cadmium absorption, which contains the DNA nucleotide sequence shown in SEQ ID NO:2, a gene encoding a transporter protein involved in the regulation of cadmium absorption, which contains the DNA nucleotide sequence shown in SEQ ID NO:3, a gene encoding a transporter protein involved in the regulation of cadmium absorption, which contains the DNA nucleotide sequence shown in SEQ ID NO:4, and a transporter protein involved in the regulation of cadmium absorption, which contains the amino acid sequence of SEQ ID NO:1.

### TECHNICAL FIELD

The present invention relates to a mutant transporter gene and a mutant transporter protein which is able to inhibit cadmium (hereinafter, abbreviated as Cd) absorption by roots, as well as rice mutants in which Cd absorption is inhibited and rice varieties with low Cd absorption.

### BACKGROUND ART

The joint FAO/WHO Codex Alimentarius Commission has established an international standard value which regulates the Cd concentration in food to reduce a risk of health damage from an intake of Cd contained in food. By the amendment of the Japanese Food Sanitation Law in February 2011 based on the international standard value, the legal limit of rice has been drastically changed from 1 mg kg⁻¹ (brown rice) to 0.4 mg kg⁻¹ (brown rice and polished rice). Therefore, it is urgent to develop a technique to decrease in Cd absorption by a rice plant.

As a technique to decrease in Cd absorption by a rice plant, agricultural techniques such as soil restoration by replacing Cd-polluted soil with non-polluted soil, inputs of soil amendments, flooding management have been carried out in the past (e.g. see Patent Literature 1 and 2). The conventional methods, however, have many problems in terms of the required number of years, costs and effects.

### CITATION LIST

### Patent Literature

Patent Literature 1: JP 2011-83194 A
Patent Literature 2: JP 2011-6529 A

### SUMMARY OF INVENTION

### Technical Problem

Development and introduction of a rice variety with low Cd absorption, which is alternative technique to the conventional methods, is economical, environmental friendly, and sustainable technique; however, there have not been reports of the development of a rice variety with low Cd absorption at all. An object of the present invention is to provide a mutant transporter protein and a mutant transporter gene which inhibit Cd absorption by roots, as well as a rice plant in which Cd absorption is inhibited and a method for selecting and raising a Cd absorption-inhibiting rice plant.

### Solution to Problem

The present inventors selected rice mutants with low Cd absorption from the rice plant library obtained by applying a heavy ion beam to rice seeds, and further identified the causative gene of the obtained mutants, thereby reaching the present invention. That is, the present invention is as follows.

<1>
<2> The present invention is a gene encoding a mutant transporter protein involved in the inhibition of cadmium absorption, which has the DNA nucleotide sequence shown in SEQ ID NO:3.
<3> Further, the present invention is a gene encoding a mutant transporter protein inhibiting cadmium absorption, which has the DNA nucleotide sequence shown in SEQ ID NO:4.
<4>
<5> Further, the present invention is a mutant transporter protein inhibiting cadmium absorption, which has the amino acid sequence of SEQ ID NO:5.
<6> Further, the present invention is a mutant transporter protein inhibiting cadmium absorption, which contains the following (P) or (R) amino acid sequences:

(P) the amino acid sequence of SEQ ID NO:6; and

(R) the amino acid sequence with a homology of at least not less than 95% to the amino acid sequence of SEQ ID NO:6, which is the amino acid sequence of a protein inhibiting cadmium absorption.

<7> Further, the present invention is a recombinant vector containing DNA according to <2> or <3> above.
<8> Further, the present invention is a transformant containing DNA according to <2> or <3> above.
<9> Further, the present invention is a transformant obtained by using the recombinant vector according to <7> above.
<10> The present invention is further a genetic marker which can identify a DNA nucleotide sequence according to <2> or <3> above.
<11>
<12> Further, the present invention is a cadmium absorption-inhibiting rice plant, in which a protein according to <5> or <6> above is expressed.
<13> Further, the present invention is the cadmium absorption-inhibiting rice plant according to <12> above, which has the same degree of ear emergence, yield and taste as of a rice variety Koshihikari.
<14>
<15>
<16> Further, the present invention is a cadmium absorption-inhibiting rice plant, which is obtained by a cross between a cadmium absorption-inhibiting rice mutant according to <12> or <13> and an existing rice variety.

### Advantageous Effects of Invention

The present invention provides a practical rice variety with low Cd absorption. Further, by using the rice variety with low Cd absorption of the present invention as mother plant, a novel rice variety with low Cd absorption can be raised without gene recombination operation. In addition, by using the DNA marker of the present invention, a rice variety with low Cd absorption can be efficiently selected.

### BRIEF DESCRIPTION OF DRAWINGS

FIG. 1 shows the cadmium concentration in brown rice when cultivation was carried out in a Cd-contaminated farm field.

FIG. 2 is explanatory diagrams showing the growth state of Koshihikari and low Cd mutant lines.

FIG. 3 is a figure showing the frequency distribution of shoot Cd concentration in F2 individuals (92 individuals) obtained by a cross between #3-6-4 and Kasalath.

FIG. 4 is a figure showing the location of a low Cd gene estimated by genetic mapping.

FIG. 5 is a figure showing the positions of deletion and insertion of nucleotides on genomic DNA in #7-3-6 and #3-6-4, respectively.

FIG. 6 is a resulting figure of electrophoresis showing differences in the length of PCR-amplified DNA fragments of wild type Koshihikari and Koshihikari mutants.

FIG. 7 is resulting figures showing the degree of proliferation of yeast when yeast mutant strains into which the TRECA gene and the treca-1 gene are introduced are treated on SD agar media.

FIG. 8 is figures of fluorescence observation showing the localities of the TRECA protein and the treca-1 protein.

FIG. 9 is a resulting figure of electrophoresis showing differences in the length of amplified DNA fragments of wild type Koshihikari and mutants (#3-6-4 and #3-5-20) using a genetic marker.

FIG. 10 is an electrophoretogram showing differences in the length of amplified DNA fragments of Koshihikari and a mutant (#7-3-6) by a genetic marker.

### DESCRIPTION OF EMBODIMENT

The present invention is a transporter protein involved in Cd absorption (Transporter regulating cadmium absorption, hereinafter abbreviated as TRECA), which is obtained by analyzing the genes of rice mutants with low Cd absorption obtained by applying a heavy ion beam, which is frequently used for flower breeding and the like, a TRECA gene and mutant genes of the TRECA gene (hereinafter, abbreviated as treca), a rice variety with low Cd absorption which contains the mutant gene, and a novel rice variety with low Cd absorption obtained by using the rice mutant with low Cd absorption. The present invention will now be described in detail.

<TRECA Gene>

The TRECA gene of the present invention is a gene which encodes the transporter protein, which is shown in SEQ ID NO:1 and is involved in cadmium absorption, and is shown in SEQ ID NO: 2. The nucleotide sequence shown in SEQ ID NO:2 is specified by analyzing the genes of a Cd absorption-inhibiting rice mutant obtained by applying the above heavy ion beam. The present invention shows an open reading frame (hereinafter, referred to as ORF) from the start codon to the stop codon of a heavy metal transporter gene derived from the TRECA gene.

In databases in which genetic information is described such as RAP-DB (The Rice Annotation Project Database) and NCBI (The National Center for Biotechnology Information), the TRECA gene is one of the Nramp genes which are annotated as genes having a heavy metal transport function by comparison with a homology to a nucleotide sequence having a known function in Arabidopsis thaliana, and the like. The TRECA gene is described as “similar to OsNramp1” in RAP-DB and “OsNramp5” in NCBI, and its function, in particular to have a function to regulate cadmium absorption in a rice plant, has not been completely known in the past.

The TRECA gene involved in the present invention is preferably an ORF having a polynucleotide having the nucleotide sequence shown in SEQ ID NO:2, and can be any gene insofar as a portion corresponding to the above portion is contained. A gene in which 5′ and 3′ UTRs are added to the ORF, for example, is also contained in the present invention.

For example, A mutant and a derivative encoding a protein having the amino acid sequence of which one or several amino acids are substituted, deleted, added and/or inserted in SEQ ID NO:1, are also included in the TRECA gene of the present invention.

In addition, even when a nucleotide sequence is mutated, there is also a possibility that the mutation is not accompanied with mutation of amino acids in a protein (degenerate mutation), and such degenerate mutants are also included in the gene of the present invention.

The method for acquiring the above gene is not particularly limited and a general method is adopted. The gene, for example, can be cut out by a proper restriction enzyme from the genomic DNA, genomic DNA library and the like of an organism having the gene and purified. That is, genomic DNA and chemically synthesized DNA are contained in the heavy metal transporter gene of the present invention. The genomic DNA can be prepared by using the usual measures of those of skill in the art. The genomic DNA can be prepared by, for example, extracting genomic DNA from a target organism, creating a genomic library (plasmid, phage, cosmid, BAC, PAC and the like can be utilized as a vector), developing this, and carrying out colony hybridization or plaque hybridization using a probe prepared based on DNA (SEQ ID NO:2) encoding the protein of the present invention.

In addition, the genomic DNA can be also prepared by creating primers specific to DNA (SEQ ID NO:2) encoding the heavy metal transporter protein of the present invention and carrying out PCR using these primers.

Specifically, the method well known by those of skill in the art for preparing a gene encoding a protein functionally equal to the heavy metal transporter protein of SEQ ID NO:1 includes a hybridization technique described in “Southern, E. M. Journal of Molecular Biology, 98, 503 (1975)”, and a method in which a polymerase chain reaction (PCR) technique is utilized, described in “Saiki, R. K. et al. Science, 230, 1350-1354 (1985), Saiki, R. K. et al. Science, 239, 487-491 (1988)”. A gene encoding a protein having a function equal to the heavy metal transporter protein of the present invention, which can be isolated by the hybridization technique and the PCR technique as described above, is also contained in the gene of the present invention.

It is believed that the gene isolated above has, at the level of amino acids encoded thereby, a high homology to the amino acid sequence (SEQ ID NO:1) of the protein of the present invention. The high homology means a sequence homology of not less than 80%, further preferably not less than 90% and particularly preferably not less than 95% to the full amino acid sequence. The sequence homology can be determined by FASTA search (Pearson W. R. and D. J. Lipman (1988) Proc. Natl. Acad. Sci. USA. 85:2444-2448) and BLASTP search.

<TRECA Protein>

The TRECA protein involved in the present invention is a protein which is encoded by the TRECA gene, exists on cell membrane and is involved in cadmium and manganese absorption. Since cadmium and manganese absorption is drastically inhibited in a mutant line (#7-2-13) in which all genes encoding the TRECA protein of the present invention described below are deleted and mutant line (#3-6-4 and #7-3-6 etc.) in which mutation is partially caused, it is obvious that the protein regulates cadmium and manganese absorption. In addition, since the TRECA protein shows the iron transport activity in a group in which the TRECA gene is introduced into yeast and does not show the iron transport activity in yeast having a mutant form, treca protein, the TRECA protein is also involved in iron absorption. The iron concentration is however not changed in the above-described deletion strain and mutant strains (Table 2), and thus the protein is expected to have a low ability to absorb iron as compared to that of iron absorption-related proteins, IRT and ZIP families (Bughio N. et al. (2002) Journal of Experimental Botany. 53: 1677-1682), which have been previously reported.

The TRECA protein involved in the present invention is a protein having the amino acid sequence shown in SEQ ID NO:1, or a protein having the amino acid sequence of which one or several amino acids are deleted, substituted or added in SEQ ID NO:1 and having the heavy metal transport function of the TRECA protein, or a protein showing a homology of not less than 80%, more preferably not less than 90% and further preferably not less than 95% at the amino acid level to a protein with the amino acid sequence shown in SEQ ID NO:1 and having the heavy metal transport function of the TRECA protein.

<Treca-1 Gene that be Mutant Type of TRECA Gene and Treca-1 Protein>

The present invention is a mutant gene of the TRECA gene (hereinafter, referred to as treca-1 gene), which is shown in SEQ ID NO:3. The nucleotide sequence shown in SEQ ID NO:3 is a gene derived from a Cd absorption-inhibiting rice mutant, #3-6-4, obtained by applying the heavy ion beam, and 32 by from nucleotide number 1025 to 1056 at the terminal portion of the 10th exon of cDNA is replaced by 50 bp in the TRECA gene shown in SEQ ID NO:2.

The present invention is an ORF from the start codon to the stop codon of the treca-1 gene shown in SEQ ID NO:3, and can be any gene in so far as a portion corresponding to the above portion is contained. A gene in which 5′ and 3′ UTRs are added to the ORF, for example, is also contained in the present invention.

For example, a mutant and a derivative encoding a protein having the amino acid sequence of which one or several amino acids are substituted, deleted, added and/or inserted in SEQ ID NO:5 are, also included in the treca-1 gene of the present invention.

In the treca-1 protein encoded by the treca-1 gene involved in the present invention, the amino acid sequence is mutated from the TRECA protein as shown in SEQ ID NO:5, and the function of Cd absorption is inhibited. The treca-1 protein involved in the present invention is a protein having the amino acid sequence shown in SEQ ID NO: 5, or a protein having the amino acid sequence of which one or several amino acids are deleted, substituted or added in SEQ ID NO:5 and lacking the heavy metal transport function of the TRECA protein, or a protein showing a homology of not less than 80%, more preferably not less than 90% and further preferably not less than 95% at the amino acid level to a protein having the amino acid sequence shown in SEQ ID NO:5 and lacking the heavy metal transport function of the TRECA protein.

<Treca-2 Gene that be Mutant Type of TRECA Gene, and Treca-2 Protein>

The present invention is a mutant gene of the TRECA gene (hereinafter, referred to as treca-2 gene), which is shown in SEQ ID NO:4. The nucleotide sequence shown in SEQ ID NO:4 is a gene derived from a Cd absorption-inhibiting rice mutant, #7-3-6, obtained by applying the heavy ion beam, and a single nucleotide deletion (cytosine deletion) is observed at the nucleotide number 915 in the TRECA gene (cDNA) shown in SEQ ID NO:2.

The treca-2 gene involved in the present invention is preferably an ORF having a polynucleotide having the nucleotide sequence shown in SEQ ID NO:4, and can be any gene insofar as a portion corresponding to the above portion is contained. For example, a gene in which 5′ and 3′ UTRs are added to the ORF, is also contained in the present invention.

For example, a mutant and a derivative encoding a protein having the amino acid sequence of which one or several amino acids are substituted, deleted, added and/or inserted in SEQ ID NO:6 are, included in the treca-2 gene of the present invention.

In the treca-2 protein encoded by the treca-2 gene involved in the present invention, the amino acid sequence is mutated from the TRECA protein as shown in SEQ ID NO:6, and the function of Cd absorption is inhibited. The treca-2 protein is a protein having the amino acid sequence shown in SEQ ID NO:6, or a protein having the amino acid sequence of which one or several amino acids are deleted, substituted or added in SEQ ID NO:6 and lacking the heavy metal transport function of the TRECA protein, or a protein showing a homology of not less than 80%, more preferably not less than 90% and further preferably not less than 95% at the amino acid level to a protein having the amino acid sequence shown in SEQ ID NO:6 and lacking the heavy metal transport function of the TRECA protein.

<Recombinant Vector of TRECA Gene and Treca Genes>

The recombinant vector by the present invention is that into which any of TRECA gene, a mutant form treca-1 gene and treca-2 gene is incorporated. By expressibly introducing the vector into a target plant by a known transformation method, a gene or a gene fragment incorporated into such plant can be expressed to obtain the protein involved in the present invention.

The recombinant vector by the present invention is preferably binary vectors, and among these, “a high capacity binary shuttle vector” described in JP H10-155485 A is particularly preferred.

<Transformant>

When a transformant expressing the gene of the present invention is created, the above vector into which the above gene is inserted is introduced into a target plant cell. A known method can be used for the introduction of the vector into the plant cell, and, for example, Agrobacterium method, electroporation method, particle gun method, microinjection method and the like are used. Among these, Agrobacterium method is the most preferred.

The above transformant expressing the gene of the present invention is not limited to rice plants, and all plants having a protein and a gene which regulate Cd absorption by roots are included in this invention.

<Genetic Marker>

The genetic marker in the present invention identifies individuals based on differences in nucleotide sequences with the above TRECA gene and mutant types, treca genes.

The above genetic marker is a mark to decide whether or not the gene of the present invention is introduced into other rice varieties by a cross and transformation based on differences in nucleotide sequences. The genomic DNA extracted from a rice plant is used as a template, and by using synthetic oligonucleotides having nucleotide sequences suitably selected in accordance with the nucleotide sequence of the genomic DNA as primers, the PCR amplification reaction is carried out in a reaction solution in which these are mixed. The reaction solution containing the product is, for example, subjected to agarose electrophoresis. The fractions of amplified DNA fragments are separated, thereby being able to confirm that a DNA fragment corresponds to the genomic DNA of the present invention.

By the present invention, for example, genomic DNA is extracted from a rice variety such as Koshihikari and a #3-6-4 cultivar. When the PCR-amplified DNA fragments of the extracts are subjected to electrophoresis, their fractions were separated into 200 to 500 bp and 600 to 800 bp depending on types of primer set which can amplify a target nucleotide portion (a portion into which nucleotides are inserted), which can be used for identifying each individual. Since an amplified DNA fragment does not appear in a #7-2-13 cultivar, the cultivar can be identified from other varieties. As all primers, those having a nucleotide sequence which can amplify a mutant region are included in this invention.

In a #7-3-6 cultivar, its DNA fragment is amplified by PCR reaction using a primer set, [e.g. [Os7g2572_F2976g (5′-TATATTCAGCCTGGGCAGATCGAG-3′: SEQ ID NO:7), Os7g2572_R3815g (5′-TGATGTACTGTCCAGCGTATGTGC-3′: SEQ ID NO:8)] or the like, which can amplify a nucleotide portion containing a single nucleotide deletion region, and the amplified DNA fragment having a restriction enzyme site newly generated by a single nucleotide deletion is subjected to cleavage treatment by a specific restriction enzyme (e.g. FspI) and the like, thereby being able to identify the mutant (#7-3-6) from other varieties. As the primers, those having a nucleotide sequence which can amplify a mutant region are not restricted. In addition, as the restriction enzymes, those which can cleave a mutant region are not restricted.

In the #7-3-6 cultivar, the amplified DNA fragment is purified by a spin column method, a glass beads adsorption method and the like, and by reading the DNA nucleotide sequence of the amplified portion with a sequencer, a single nucleotide deletion contained in the DNA nucleotide sequence of the present invention is detected. By developing a SNP (single nucleotide polymorphism) marker based on this information, its individual can be identified.

<Development of Cadmium Absorption-Inhibiting Rice Mutants Utilizing a Heavy Ion Beam>

A heavy ion beam is applied to rice seeds. The dose of a heavy ion beam for the development of cadmium absorption-inhibiting rice mutants are not particularly limited within a range in which rice seeds are not damaged and mutation can be induced. When a carbon ion beam which can induce mutation with high frequency is used, a range from 20 to 60 Gray is preferred.

The seeds to which the above heavy ion beam is applied (first generation mutant seeds, hereinafter abbreviated as M1, and the same applies to the second generation and the subsequent generation) are used for common cultivation, and the obtained M2 seeds are used for the selection of low Cd mutants. As methods for selecting low Cd mutants using M2 seeds, there are the following two methods.

For the selection of M2, a method for selecting low Cd mutants by treating 10-day old seedlings after sowing with a Cd-containing hydroponic solution, then cutting the stems of the seedlings, and measuring Cd concentrations in a xylem vessel liquid exuded from the cut surface with an atomic absorption photometer or the like is particularly desirable when there is only a reduced space such as a culture room and a Cd-contaminated soil cannot be obtained. In addition, next generation seeds (M3 seeds) can be secured by growing buds (tillers) emerged from stubble after cutting. Further, rice plants with low Cd absorption, which are the subsequent generation such as M4 and M5, can be obtained by self-fertilization of the seeds (M3 seeds).

When a Cd-contaminated soil can be utilized, the seedlings passed for a month from sowing of M2 seeds in artificially fertile soil are transplanted into a Cd-contaminated soil, and the Cd concentration in brown rice obtained by going through the steps of harvesting, drying, threshing and hulling is analyzed. A rice plant with low Cd absorption (M3 seeds) can be obtained by selecting a individual with low Cd concentration in brown rice. As with the hydroponic cultivation, by self-fertilization of the seeds, rice plants with low Cd absorption, which are the subsequent generation such as M4 and M5, can be obtained.

<Acquisition of a Heavy Metal Transporter Gene from a Rice Plant with Low Cd Absorption>

The methods for acquiring the genes involved in the present invention include a hybridization technique and a polymerase chain reaction (PCR) technique. In the former, for example, a probe which specifically hybridizes with the full or partial nucleotide sequence of a gene in the present invention is prepared and a genomic DNA library and a cDNA library are screened. In the latter, for example, using known sequence information of Nipponbare, a primer set (5′ side and 3′ side) to amplify a gene of the present invention by a PCR method is designed, and PCR is carried out using cDNA as a template, and the gene involved in the present invention can be acquired by amplifying a DNA region between both primers.

<Identification of a Heavy Metal Transporter Protein>

As the protein involved in the present invention, the nucleotide sequence of the gene isolated above is determined by the cycle sequence method, and the nucleotide sequence can be translated into an amino acid sequence according to codons. The protein can be identified by comparing the amino acid sequence with rice genome database (RAP-DB).

<Method for Breeding of a Novel Rice Plant with Low Cd Absorption Using a Rice Mutant with Low Cd Absorption as Mother Plant>

Using the genetic marker by the present invention, a novel rice variety with low Cd absorption can be efficiently bred by a cross between a rice mutant with low Cd absorption having DNA obtained by the present invention and an existing variety.

As the breeding method, there are the following procedures:

1) F1 is bred by a cross between a rice mutant with low Cd absorption (Plant A) and an existing rice variety (Plant B);
2) the F1 and the Plant B are crossed;
3) individuals having a low Cd gene are selected from plants after crossing by using a genetic marker;
4) the low Cd gene of Plant A (e.g. treca-1 gene or treca-2 gene) is intentionally introduced into Plant B by “backcrossing” which is repeated crossing with Plant B;
5) in this case, to select only an individual having the low Cd gene among a large number of backcrossed individuals, a genetic marker can be utilized, and by the genetic marker of the present invention, an individual having the low Cd gene can be marked off from the other individuals;
6) when the individual above is selected, genomic DNA extracted from shoots and roots in the seedling stage can be used;
7) a novel variety (e.g. low Cd Akitakomachi) in which most of the genome structure is Plant B and only Cd absorption is the inherited character of Plant A can be bred by repeating crossing with Plant B and selecting only an individual having the low Cd gene with a marker; and
8) Plant B may be either Japonica rice or Indica rice.

### EXAMPLES

The contents of the present invention will now be described in more detail by way of examples. It should be noted, however, that the present invention is not limited to the description of the examples.

### Example 1

### Selection of Rice Mutants with Reduced Cd Absorption

(Irradiation of a Heavy Ion Beam)

Using TIARA of Takasaki Advanced Radiation Research Institute, Japan Atomic Energy Agency, 3500 grains of rice plant (Koshihikari cultivar) seeds (the present seeds are first generation seeds, hereinafter abbreviated as M1, and the second, third generation and the like are referred to as M2, M3 and the like) to which a heavy ion beam (carbon ion, 320 MeV, 40 Gy) was applied were seeded in compost (manufactured by Sumitomo Chemical Co., Ltd., Product Name: Bonsoru 1). The obtained seedlings were transplanted as each individual in a paddy farm field possessed by National Institute for Agro-Environmental Sciences and M2 seeds were obtained from each individual by conventional cultivation management in National Institute for Agro-Environmental Sciences. The obtained M2 seeds, approximately 100,000 grains, all were mixed and used for the following selection step of low Cd mutants.

(First Selection Method of Low Cd Mutants—Simple Selection Method Using Xylem Vessel Cd Concentration as an Index)

The germinated seeds of M2 were seeded in a 96 well PCR plate with holes with a diameter of 3 mm on the bottom surface thereof. The plate was put on a floating stand produced using styrofoam and the stand was put in a 20 L container with Kimura B hydroponic solution (1/2 concentration).

The seedlings after a lapse of 10 days from sowing were treated with a hydroponic solution with a Cd concentration of 0.1 ppm for 4 days. After the treatment, the stem part of each individual was cut at 2 cm above the plate and a xylem vessel liquid bled from the cut surface was permeated into absorbent cotton, and only the xylem vessel liquid was collected by centrifugation (2,000 rpm, 1 min). The plate for collection was obtained by boring holes with a diameter of 2 mm on the bottom of a 96 well PCR plate, filling absorbent cotton therein, and then putting a plate without holes thereon. A xylem vessel liquid was permeated into cotton and centrifugation was carried out (2,000 rpm, 1 min), thereby collecting the liquid in the lower plate.

The xylem vessel liquid collected above was diluted 50 to 200-fold with 0.1M nitric acid and the Cd concentration was measured by an atomic absorption photometer (manufactured by Agilent Technologies, Product Name; spectraAA 220Z). Only individuals showing a Cd concentration which was 1/5 or less of the Cd concentration in a xylem vessel liquid collected from Koshihikari were collected and tillers (buds emerged from stubble) were grown and transplanted in compost to secure next generation seeds (M3) and the seeds were collected.

Approximately 3,000 individuals were subjected to Cd treatment by the above method. Among these, 4 mutants (#3-5-20, #6-4-10, #11-6-12, #12-3-5) were selected, in which growth was equal to that of Koshihikari and the xylem vessel Cd concentration was 1/5 to 1/20 of the concentration of Koshihikari, and M3 seeds were collected by the above method. The xylem vessel Cd concentrations in the mutants are shown in Table 1. In Table 1, Koshihikari and a mutant cultivated on the same plate were individually compared.

[Table 1]

(Second Selection Method of Low Cd Cultivars—Selection Method Using Cd Concentration in Brown Rice as an Index)

The selection method using the Cd concentration in a xylem vessel liquid as an index can be easily carried out in a reduced space, however, the method cannot evaluate whether or not a brown rice individual with low Cd concentration is selected. Therefore, for the purpose of selecting mutants with low Cd concentration in brown rice, a method for directly analyzing the Cd concentration in brown rice is preferred; however, for cultivating a large number of cultivars in a Cd-contaminated farm field until the filling stage, a specific place is required. Herein, a method was devised, by which a large number of cultivars can be cultivated when there are only a small amount of Cd-contaminated soil and a relatively reduced space such as a greenhouse and low Cd mutants can be selected by the Cd concentration in brown rice.

The seedlings, 2592 individuals, passed for a month after sowing the germinated seeds of M2 were transplanted as each individual in a small-sized polyethylene flowerpot (Y pot, a diameter of 7.5 cm, holes on the bottom surface) filled with a Cd-contaminated soil (the Cd concentration of soil extracted with 0.1 M hydrochloric acid: 1.8 mg kg⁻¹) and 288 individuals of Koshihikari untreated with a heavy ion beam were transplanted in the same manner as a control. In a plastic container (new TO tray), 40 pots each were separated and placed and flooding was managed until the boot stage. After that, Cd was eluted from the soil by surface drainage and tap water was constantly provided until the filling stage in the extent to which the soil was not dried. For fertilization, 0.05 g-N/pot (300 g of soil) was provided in the maximum tillering stage. Harvesting was carried out in each individual, and brown rice of the M3 generation was obtained by going through the steps of drying, threshing and hulling. The obtained M3 brown rice was completely digested by nitric acid-perchloric acid and the digestion liquid was suitably diluted with mill-Q water, and the Cd concentration in the diluted solution was then measured by an inductively coupled plasma mass spectrometer (manufactured by PerkinElmer, Product Name; ELAN DRC-e).

By the above method, three brown rice cultivars with low Cd concentration in M3 seeds (#3-6-4, #7-3-6, #7-2-13) were selected.

### Example 2

### Confirmation of the Low Cd Trait of Mutants

(Evaluation in Hydroponic Cultivation)

The seeds of M3 lines of low Cd mutants (#3-6-4, #7-3-6, #7-2-13) selected using the Cd concentration in brown rice as an index and Koshihikari untreated with a heavy ion beam (hereinafter, simply referred to as Koshihikari) were seeded and the seedlings after a lapse of 10 days as with Example 1 were treated with Cd by a hydroponic method. After a lapse of 4 days from Cd treatment, harvesting was carried out, and the harvest was separated into shoots and roots, followed by drying. The dry samples were digested with a strong acid by the same method as for the brown rice in Example 3. In addition to Cd, manganese (Mn), copper (Cu), iron (Fe) and zinc (Zn), essential elements for plants, were contained in the decomposition liquid and all of the elements were simultaneously measured by an inductively coupled plasma optical emission spectrometer (ICP-OES) (manufactured by Agilent Technologies, Product Name; Vista-Pro). The results are shown in Table 2.

[Table 2]

From Table 2, all of the shoot Cd concentration in the mutant strains was approximately 1/7 as compared to that of Koshihikari. In addition, the root Cd concentration was approximately ¼ as compared to that of Koshihikari in all mutant strains. This found that low Cd in all mutant strains was caused by low Cd absorption of roots. As the results of the comparison with other heavy metal concentrations, the mutant strains were characterized by remarkably low manganese concentrations in both shoots and roots and the same phenomenon was observed in all mutant strains.

(Evaluation in a Cd-Contaminated Farm Field)

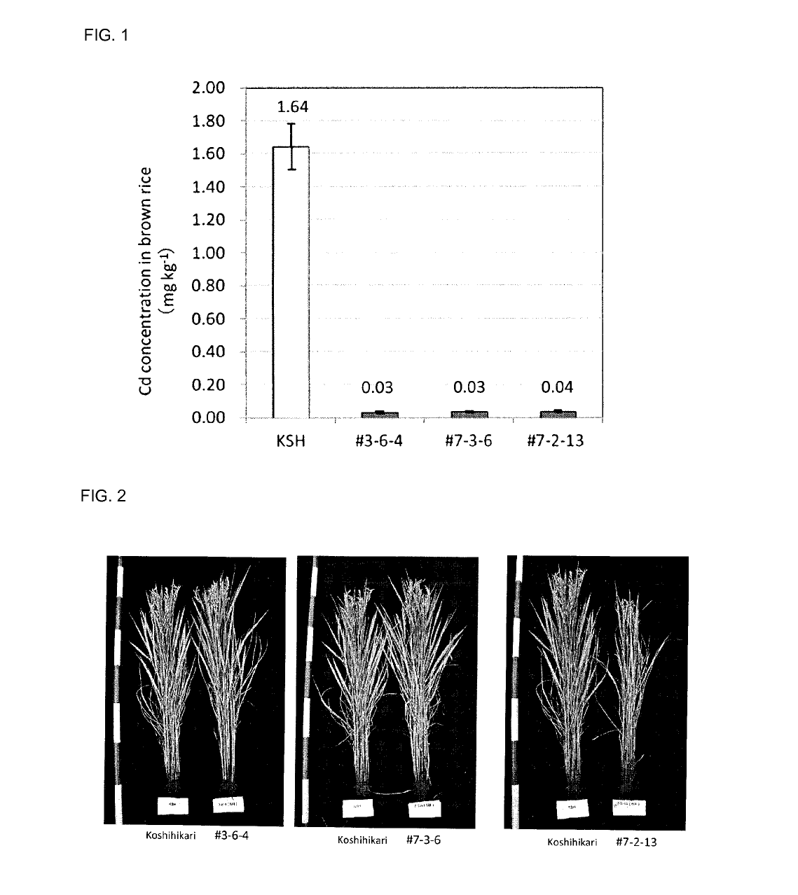

The subsequent generation, M4 strain, of the low Cd mutants (#3-6-4, #7-3-6, #7-2-13) selected by the Cd concentration in brown rice in Example 1, and Koshihikari were cultivated in a Cd-contaminated farm field (Cd concentration 1.8 mg kg⁻¹). Water was managed by midseason drainage and intermittent irrigation according to a conventional method of paddy-rice cultivation. For fertilization, 5 kg-N/10 a, 8 kg-P205/10 a and 8 kg-K20/10 a were provided as initial manure and 2 kg-N/10 a was applied as ear manuring. Brown rice in the filling stage was harvested and decomposed by a strong acid. After that, Cd contained in the solution was measured by ICP-MS (manufactured by PerkinElmer, Product Name; ELAN DRC-e) and other heavy metals were measured by ICP-OES (manufactured by Agilent Technologies, Product Name; Vista-Pro). The results were shown in FIG. 1 and the growth state during cultivation was shown in FIG. 2.

From FIG. 1, the Cd concentration in M5 brown rice from the three mutant strains was remarkably low as compared to that of Koshihikari and it was found that a low Cd trait in mutants was stably low even in brown rice of advanced generation. Further, as with (Evaluation in hydroponic cultivation), the manganese concentration was also low in brown rice, which was not more than half that of Koshihikari. The concentration of zinc, copper and iron in brown rice from the mutant strains was the same degree as of Koshihikari.

From FIG. 2, in one (#7-2-13) among the mutants selected in Example 1, ear emergence was two weeks earlier and the height was shorter than those of Koshihikari. In the other mutant strains, the ear emergence stage was equal to that of Koshihikari and the strains could not be distinguished from Koshihikari at their appearance. The influence on leaf color due to the low manganese concentration, sterility and the like were not observed at all.

### Example 3

### Genetic Analysis

(Microarray Experiments)

RNA was extracted from roots of the above mutant (#3-6-4) and Koshihikari according to the protocol of an RNA purification kit (manufactured by Rizo Inc., Product Name: RNAs-ici!-S). The RNA concentration was measured by a spectrophotometer (manufactured by Thermo Fisher Scientific, Product Name: NanoDrop 1000) and the quality was checked by Agilent 2100 Bioanalyzer to confirm whether or not the extracted RNA was degraded.

The extracted total RNA (400 ng) was reversely transcribed using T7 promoter primer (Agilent) to synthesize cDNA. For fluorescence labeling, Cyanine 3 (hereinafter, referred to as Cy3) and Cyanine 5 (hereinafter, referred to as Cy5) were added thereto and a labeled cRNA was synthesized by reaction in Transcription Mix solution with T7 RNA polymerase (Agilent).

The labeled cRNA extracted and synthesized from each individual described above was purified by Qiagen RNeasy Kit (manufactured by Qiagen) and the RNA concentration and quality were then checked using the above Agilent 2100 Bioanalyzer. The cRNA labeled with Cy3 and the cRNA with Cy5 were mixed in a target cRNA solution and a fragmentation buffer (Agilent) was added thereto for fragmentation of the target, followed by incubation at 60° C. for 30 minutes. The solution was added dropwise to the probe surface of the rice oligo DNA microarray 4×44 RAP-DB (manufactured by Agilent) on which 43,803 of synthetic oligonucleosides were placed, and the surface was covered with a cover glass, followed by the hybridization reaction in a constant temperature oven (60° C., 17 hours, 10 rpm).

The above cover glass was rinsed with two types of cleansing liquid, and water droplets were removed by nitrogen gas, and fluorescent signals were measured using a microarray scanner (manufactured by Agilent). Feature Extraction software from Agilent was used for the quantification of fluorescent signals. The working process on microarray was carried out according to Text for Microarray Experiment and Analysis (edited and published by National Institute of Agrobiological Sciences).

In the above low Cd mutant (#3-6-4) selected in Example 2, it was believed that a transporter carrying cadmium and manganese into cells was mutated. Therefore, in the results of the above microarray experiments, the gene expression of the Nramp family was particularly focused, wherein the Nramp family has been reported in Arabidopsis thaliana (Thomine et al., 2000, PNAS, 97, 4991-4996) and yeast (Cohen et al., 2000, J. Biol. Chem., 275, 33388-33394) thus far and has been identified as a transporter of divalent cations (Fe²⁺ and Mn²⁺ etc.). As the rice Nramp family, OsNramp1 to OsNramp7 have been found (Takahishi et al., 2011, J. Exp. Bot., 62, 4843-4850). Among these, as the gene expression of OsNramp5 (Os07g0257200), a 2.5-fold difference in expression between a mutant and Koshihikari was observed and significant differences in expression were not observed in the other genes in the Nramp family (a 1.0 to 1.7-fold difference).

(Genetic Mapping)

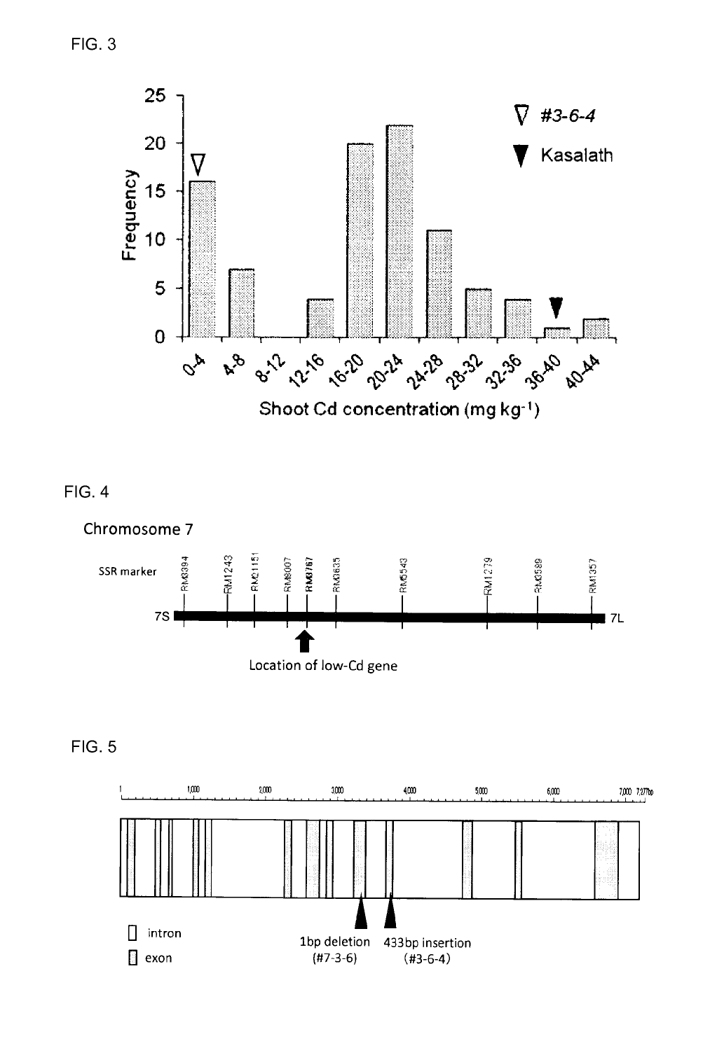

To identify a low Cd absorption gene of the low Cd mutant (#3-6-4), genetic mapping was carried out. The F2 seeds obtained by a cross between #3-6-4 and Kasalath, an indica variety, were seeded, and 92 individuals of seedlings were cultivated in Kimura B hydroponic solution with a Cd concentration of 0.02 ppm (1/2 concentration) for 4 days, and the shoot Cd concentration in each individual was measured. The Cd concentration was classified at intervals of 4 mg kg⁻¹, and the results were shown in FIG. 3.

From FIG. 3, a shoot Cd concentration of 8-12 mg kg⁻¹ was considered as a boundary and two groups existed. Among 92 individuals, 22 individuals were in the low Cd group and the remaining 70 individuals were in the high Cd group, and the segregation ratio was 1:3 (χ2=0.058, p=0.810). This found that low Cd mutants were regulated by a single recessive gene.

Next, to identify (mapping) the chromosomal location of a recessive gene, the genotypes of 92 individuals were examined utilizing 97 microsatellite (SSR) markers. A linkage map was established by software of MAPMAKER/EXP ver. 3.0. The genetic mapping was carried out by software (QTL Cartographer ver. 2.5) based on the Cd concentration and genotype data of 92 individuals.

The result of genetic mapping was shown in FIG. 4. From FIG. 4, it was found that a low Cd gene existed near an SSR marker RM3767 on chromosome 7. As the genetic search results by RAP-DB, OsNramp5 annotated as a heavy metal transporter gene existed near this marker; however, the function of OsNramp5, for example which heavy metal element was transported thereby, was not clear.

(Determination of a Nucleotide Sequence)

From the results of the above (microarray experiments and genetic mapping), OsNramp5 was focused as a target, and the presence or absence of the insertion of mutation was confirmed. RNA was extracted from roots of the cultivars (#3-6-4, #7-3-6, #7-2-13) and Koshihikari with the above RNA purification kit (RNAs-ici!-S), and after that, a single-stranded cDNA was synthesized by a reverse transcriptase (manufactured by TOYOBO CO., LTD., Product Name: ReverTra Ace). The cDNA was used as a template and PCR was carried out using a primer set, CNPorf5 (5′-CAC CAT GGA GAT TGA GAG AGA GAG CAG TG-3′: SEQ ID NO:9) and CNPrt3 (5′-ACA CCC TTG TCG ATC GAT CGA TCT G-3′: SEQ ID NO:10) (manufactured by Operon Biotechnologies K.K.) to clone an amplified fragment containing the full length ORF into pENTR/D-TOPO vector (manufactured by Invitrogen). Using Universal M13 sequencing site [M13 Forward (−20) (5′-GTA AAA CGA CGG CCA G-3′: SEQ ID NO:11), M13 Reverse (5′-CAG GAA ACA GCT ATG AC-3′: SEQ ID NO:12)] and a TRECA gene specific primer [CNP_GcheckFW (5′-GCA AGT CGA GTG CGA TCG TG-3′: SEQ ID NO:13), CNP_GcheckRV (5′-CGC CGA TGA TGG AGA CGA TG-3′: SEQ ID NO:14)] contained in the vector, the nucleotide sequence of the TRECA gene cloned into pENTR/D-TOPO vector (manufactured by Invitrogen) was determined by a DNA sequencer ABI3130xl (manufactured by Applied Biosystems).

In addition, to decode the genomic sequence of a candidate gene, genomic DNA was extracted according to a method by Xu et al. (2005, Plant Molecular Biology Reporter 23, 291-295). The primers on both sides of a region in which mutation in an ORF sequence was observed were designed [CNP_GcheckFW (5′-GCA AGT CGA GTG CGA TCG TG-3′: SEQ ID NO:13), CNP_GcheckRV (5′-CGC CGA TGA TGG AGA CGA TG-3′: SEQ ID NO:14)] based on the database of RAP-DB, and genomic DNA was amplified by PCR, and the nucleotide sequence was then determined by direct sequence analysis.

In the nucleotide sequence determined above, Nipponbare and Koshihikari showed the exactly same sequence and the sequence was shown in SEQ ID NO:2. The ORF sequence of the mutant (#3-6-4) was shown in SEQ ID NO:3, and the ORF sequence of the mutant (#7-3-6) was shown in SEQ ID NO:4.

The length of the ORF of Koshihikari/Nipponbare was 1617 bp. In the ORF of #7-3-6, a single nucleotide deletion (deletion of cytosine) was observed at position 915 in the sequence of Koshihikari/Nipponbare. In addition, in the ORF of #3-6-4, 32 bp from position 1025 to 1056 in the ORF of Koshihikari/Nipponbare was changed to the insertion of 50 bp and the length of ORF was changed to 1635 bp.

In #7-2-13, a PCR-amplified product was not obtained, and thus it was decided that the nucleotide sequence of the candidate gene was almost deleted.

The amino acid sequence of Koshihikari/Nipponbare was shown in SEQ ID NO:1. In #7-3-6, a frame shift (a reading frame shift) was caused due to a single nucleotide deletion to drastically change amino acids after position 306, and since the stop codon appeared earlier than that of Koshihikari, translation was stopped at position 358. The amine acid sequence of the mutant (#7-3-6) was shown in SEQ ID NO:6.

In addition, in #3-6-4, 11 amino acids from position 341 to 352 (TGTYAGQYIMQ) were replaced by 17 amino acids (RPVTMGVSLVCHAHLIG) due to the insertion of nucleotides. The reading frame shift was not caused even by the insertion and amino acids after that were the exactly same as of Koshihikari. The amino acid sequence of the mutant (#3-6-4) was shown in SEQ ID NO:5.

Further, the positions of nucleotide deletion in #7-3-6 and nucleotide insertion in #3-6-4 on genomic DNA were schematically shown in FIG. 5. In #7-3-6, cytosine (C) at position 138 in the 9th exon was deleted. In #3-6-4, the insertion of nucleotides of 433 bp was observed after adenine (A) at position 73 in the 10th exon. In the nucleotides of 433 bp, 50 bp was inserted into the exon and the remaining 383 bp was inserted into an intron. The inserted 433 bp was a transposon (transposable element), named mPingA1.

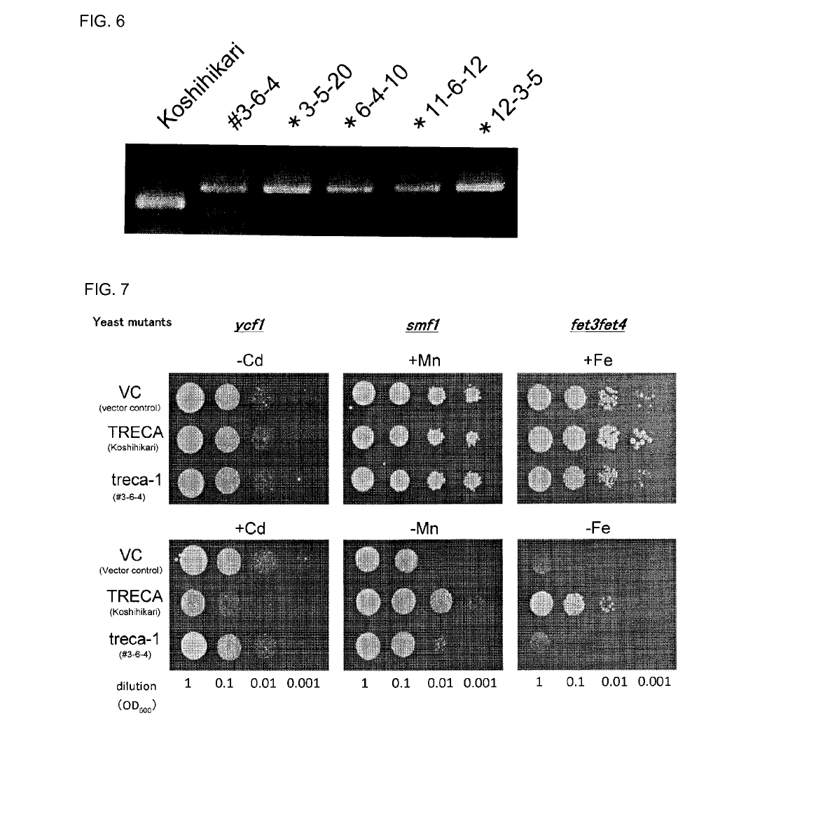

In addition, in all of four low Cd mutants (#3-5-20, #6-4-10, #11-6-12, #12-3-5) selected using the Cd concentration in a xylem vessel liquid as an index, the insertion of the same position and the same number of nucleotides as of #3-6-4 was observed, and when comparing to the length of a PCR-amplified DNA fragment of a mutant point, these individuals had the same length as of #3-6-4. The differences in the length of PCR-amplified DNA fragments of the above mutants and Koshihikari were shown in FIG. 6.

Based on these results, a protein encoded by the OsNramp5 gene was expected to regulate Cd absorption, and thus the OsNramp5 gene derived from Koshihikari was named TRECA (Transporter regulating cadmium absorption) gene, and mutant forms thereof were named treca-1 (derived from #3-6-4) gene, and treca-2 (derived from #7-3-6) gene and treca-3 (derived from #7-2-13) gene.

### Example 4

### Functional Analysis Using Yeast

Using cDNA of Koshihikari and a mutant (#3-6-4) as a template, PCR was carried out using a primer set, CNPorf5 (5′-CAC CAT GGA GAT TGA GAG AGA GAG CAG TG-3′: SEQ ID NO:9) and CNPrt3 (5′-ACA CCC TTG TCG ATC GAT CGA TCT G-3′: SEQ ID NO:10) (manufactured by Operon Biotechnologies K.K.) to clone an amplified fragment containing the full length ORF into pENTR/D-TOPO.

Further, a multicloning site was inserted into a gateway vector for yeast expression, pDR195 (Rentsch et al., 1995), by LR reaction. The constructs and pDR195 (vector control) were each introduced into (1) a Cd sensitivity mutant strain, Δycf1 (MATalpha trp1-63 leu2-3, 112 gcn4-101 his3-609 ura3-52 ycf1::TRP1), (2) a Mn requiring mutant strain, Δsmf1 (MATa, his3Δ1; leu2Δ0; met15Δ0; ura3Δ0; YOL122c::kanMX4) and (3) Fe requiring mutant strain, Δfet3fet4 (MATa/MAT alpha ade2/+can1/can1 his3/his3 leu2/leu2 trp1/trp1 ura3/ura3 fet3-2::His3/fet3-2::HIS3 fet4-1::LEU2/fet4-1::LEU2) by lithium acetate.

After culturing in SD liquid culture medium in accordance with amino acid requirement of each yeast, dilution series (OD₆₀₀=1, 0.1, 0.01, 0.001) of normal SD agar medium (—Cd, +Mn, +Fe) or SD agar medium (1) with 10 mM CdCl₂ (+Cd), (2) with 10 mM EGTA without Mn (—Mn), and (3) with 10 mM BPDS (—Fe) were created and yeast was spotted thereto, followed by culturing at 30° C. for 3 days. The TRECA gene and the treca-1 gene each were introduced into the Cd sensitivity strain (Δycf1), the Mn requiring mutant strain (Δsmf1) and the Fe requiring mutant strain (Δfet3fet4) for ±Cd, ±Mn, ±Fe treatment. From the degree of proliferation of each mutant yeast strain, differences in the ability to transport heavy metals of the proteins encoded by two genes were shown in FIG. 7.

From FIG. 7, in the strain into which the TRECA gene was introduced, proliferation was low (particularly OD₆₀₀=0.1) as compared to that of the vector control (VC) and thus Cd sensitivity was increased. This means that Cd is absorbed into yeast and yeast cannot proliferate by the influence of Cd toxicity. On the other hand, since the ability to absorb Cd was deleted in the Δycf1 strain into which the treca-1 gene was introduced, the influence of toxicity by Cd was low and proliferation almost equal to that of VC was shown. The Δsmf1 is a yeast strain which lacks an ability to transport Mn. In the strain into which the TRECA gene was introduced, higher proliferation (OD₆₀₀=0.01) than that of VC was observed particularly in the —Mn treatment zone. On the other hand, in the strain into which the treca-1 gene was introduced, proliferation was the same degree as of VC. This was believed that Mn absorption was recovered by the introduction of the TRECA gene. The Δfet3fet4 is a yeast strain which lacks an ability to transport iron. In both +Fe and —Fe treatment zones, higher proliferation than that of VC was shown in the yeast strain into the TRECA gene was introduced. On the other hand, in the strain into which the treca-1 gene was introduced, proliferation was the same degree as of VC. This revealed that the TRECA protein derived from Koshihikari had a function to transport Cd, Mn and Fe and the treca-1 protein derived from #3-6-4 lost the activity to transport Cd, Mn and Fe.

### Example 5

### Confirmation of Locality of a Causative Gene

Using cDNA of Koshihikari and a cultivar thereof (#3-6-4) as a template, PCR was carried out using a primer set, CNPorf5 (5′-CAC CAT GGA GAT TGA GAG AGA GAG CAG TG-3′: SEQ ID NO:9) and CNPorf3 (5′-CCT TGG GAG CGG GAT GTC GGC CAG G-3′: SEQ ID NO:10) (manufactured by Operon Biotechnologies K.K.) to amplify an ORF region.

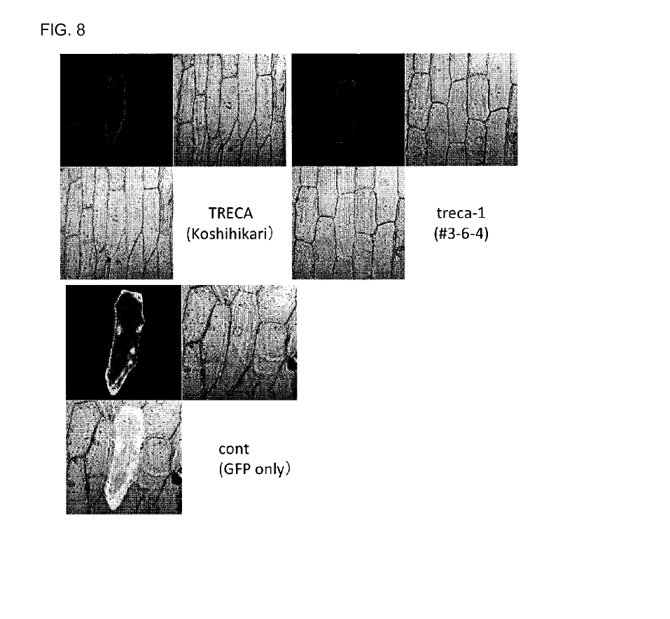

The amplified fragment was cloned into pENTR/D-TOPO (manufactured by invtrogen) to obtain each entry clone. By LR reaction using LR clonase II (manufactured by invtrogen), each ORF was inserted into pH7FWG2,0 (Karimi et al., 2002). The created construct was introduced into the onion epidermal cells by a particle bombardment method and the cells were left to stand under darkness. After 5 to 6 hours, fluorescence was observed using LSM5 Pascal laser-scanning confocal microscope (Carl Zeiss). The results are shown in FIG. 8.

From FIG. 8, fusion proteins, in which GFP was linked to the C-terminal side of the TRECA protein derived from Koshihikari and the treca-1 protein derived from the #3-6-4 strain, were expressed on the onion epidermal cells and the intracellular localization of the TRECA protein and the treca-1 protein was examined by observing green fluorescence of GFP. Both proteins were localized in cell membrane. In addition, a great difference in fluorescence strength was not observed between both proteins.

### Example 6

### Method for Distinguishing a Low Cd Rice Plant by a DNA Marker

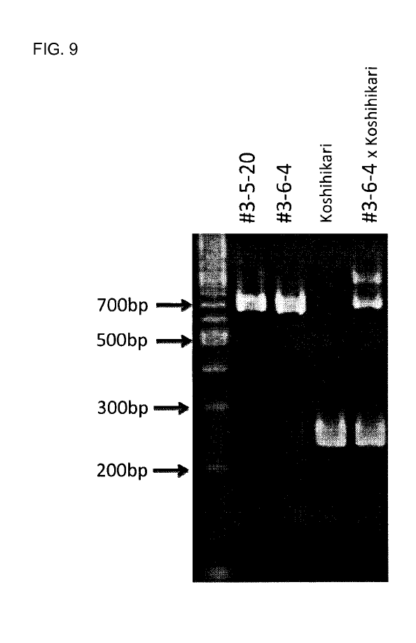

With regard to the above mutant (#3-5-20), mutant (#3-6-4), Koshihikari and an F1 individual of the cultivar (#3-6-4) and Koshihikari, genomic DNA was extracted from roots or leaves thereof and DNA levels were measured by a spectrophotometer (manufactured by Thermo Fisher Scientific, Product Name: NanoDrop 1000). Based on the nucleotide sequence data in Example 3, a primer set on both sides of a point in which mutation was inserted was designed [Os7g2572_F3711g (5′-TTC AGA ACG TGC TGG GCA AGT CG-3′: SEQ ID NO:11), Os7g2572_R3951g (5′-ACG GAT TAA CAA ATT AAT TAT GTG GCA G-3′: SEQ ID NO:12)]. Using KAPA2G Fast PCR kit (manufactured by KAPA BIOSYSTEMS), a DNA fragment was amplified by PCR using genomic DNA as a template. The obtained PCR product was put on a 3% agarose gel and electrophoresis was carried out. The results were shown in FIG. 9.

In FIG. 9, Koshihikari had an amplified DNA fragment at 240 bp, while #3-5-20 and #3-6-4 had an amplified DNA fragment around 700 bp and thus Koshihikari and the mutants with insertion could be distinguished. The F1 hetero individual of #3-6-4 and Koshihikari can be also distinguished and can be utilized as a co-dominance marker.

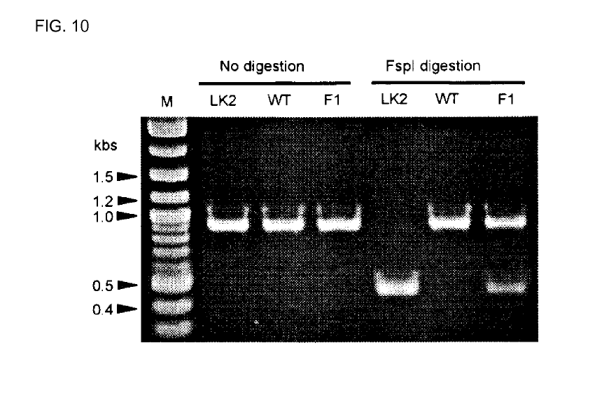

With regard to the above mutant (#7-3-6), Koshihikari, and an F1 individual of the mutant (#7-3-6) and Koshihikari, genomic DNA was extracted by the same method as above. A primer set on both side of a point in which a single nucleotide was deleted was designed [Os7g2572_F2976g (5′-TATATTCAGCCTGGGCAGATCGAG-3′: SEQ ID NO:7), Os7g2572_R3815g (5′-TGATGTACTGTCCAGCGTATGTGC-3′: SEQ ID NO:8), and a DNA fragment was amplified by PCR reaction using KAPA2G Fast PCR kit. The obtained PCR product was cleaved by a restriction enzyme, FastDigest FspI (manufactured by Thermo Scientific). The cleaved PCR product was put on a 1% agarose gel and electrophoresis was carried out. In addition, PCR products untreated with FspI were also subjected to electrophoresis for comparison. The results were shown in FIG. 10. In FIG. 10, M, LK2, WT and F1 show a size marker, #7-3-6, Koshihikari and #7-3-6×Koshihikari, respectively.

When untreated with FspI, all of #7-3-6, Koshihikari and the F1 individual had an amplified fragment at around 839 bp, and the band patterns of the three were not apparently different. Since the DNA fragment of Koshihikari did not have a recognition site of nucleotides to be cleaved by FspI, the band pattern was the same as of untreated samples. On the other hand, in #7-3-6, a new recognition site which can be cleaved by FspI was generated due to a single nucleotide deletion and 839 bp was cleaved into 419 bp and 420 bp. Since the length after cleavage was the almost same, two bands were overlapped and present as a band on the gel. The F1 hetero individual of #7-3-6 and Koshihikari can be distinguished in the same technique.

### INDUSTRIAL APPLICABILITY

By the present invention, there is provided a practical, epoch-making variety with low Cd absorption. Furthermore, a novel variety with low Cd absorption can be raised without a gene recombination operation by using the mutant with low Cd absorption of the present invention as mother plant, and an individual with low Cd absorption can be efficiently selected by using the DNA marker of the present invention.

1. (canceled)

2. A gene encoding a transporter protein inhibiting cadmium absorption, which has the DNA nucleotide sequence shown in SEQ ID NO:3.

3. A gene encoding a transporter protein inhibiting cadmium absorption, which has the DNA nucleotide sequence shown in SEQ ID NO:4.

4. (canceled)

5. A mutant transporter protein inhibiting cadmium absorption, which has the amino acid sequence of SEQ ID NO:5.

6. A mutant transporter protein inhibiting cadmium absorption, which contains either the following (P) or (R) amino acid sequences:
(P) the amino acid sequence of SEQ ID NO:6;
(R) the amino acid sequence with a homology of at least not less than 95% to the amino acid sequence of SEQ ID NO:6, which is the amino acid sequence of a protein regulating cadmium absorption.

7. A recombinant vector containing DNA according to claim 2.

8. A transformant containing DNA according to claim 2.

9. A transformant obtained by using the recombinant vector according to claim 7.

10. A genetic marker for specifying an individual containing a DNA according to claim 2.

11. (canceled)

12. A cadmium absorption-inhibiting rice plant, in which a protein according to claim 5 is expressed.

13. The cadmium absorption-inhibiting rice plant according to claim 12, which has the same degree of ear emergence, yield and taste as of a rice variety Koshihikari.

14. (canceled)

15. (canceled)

16. A cadmium absorption-inhibiting rice plant, which is obtained by a cross between a cadmium absorption-inhibiting rice mutant according to claim 12 and an existing rice variety.

17. A recombinant vector containing DNA according to claim 3.

18. A transformant containing DNA according to claim 3.

19. A genetic marker for specifying an individual containing a DNA according to claim 3.

20. A cadmium absorption-inhibiting rice plant, in which a protein according to claim 6 is expressed.

21. A cadmium absorption-inhibiting rice plant, which is obtained by a cross between a cadmium absorption-inhibiting rice mutant according to claim 13 and an existing rice variety.

22. A transformant obtained by using the recombinant vector according to claim 17.


SEQUENCE LISTING (14 sequences)

SEQ ID NO: 1 - protein, 538 aa, Oryza sativa
MEIERESSERGSISWRASAAHDQDAKKLDADDQLLMKEPAWKRFLAHVGPGFMVSLAYLDPGNLETDLQAGANHRYELLWVILIGLIFALIIQSLAANLGVVTGRHLAEICKSEYPKFVKIFLWLLAELAVIAADIPEVIGTAFAFNILFHIPVWVGVLITGTSTLLLLGLQKYGVRKLEFLISMLVFVMAACFFGELSIVKPPAKEVMKGLFIPRLNGDGATADAIALLGALVMPHNLFLHSALVLSRKTPASVRGIKDGCRFFLYESGFALFVALLINIAVVSVSGTACSSANLSQEDADKCANLSLDTSSFLLKNVLGKSSAIVYGVALLASGQSSTITGTYAGQYIMQGFLDIRMRKWLRNLMTRTIAIAPSLIVSIIGGSRGAGRLIIIASMILSFELPFALIPLLKFSSSKSKMGPHKNSIYIIVFSWFLGLLIIGINMYFLSTSFVGWLIHNDLPKYANVLVGAAVFPFMLVYIVAVVYLTIRKDSVVTFVADSSLAAVVDAEKADAGDLAVDDDEPLPYRDDLADIPLPR

SEQ ID NO: 2 - DNA, 1617 bp, Oryza sativa
atggagattgagagagagagcagtgagagagggagcatcagctggagagctagtgcggcacatgatcaagatgccaagaagctcgacgcagatgatcagctgctaatgaaggagcctgcatggaaaaggttccttgcccatgttggtcctggattcatggtgtctttagcctacttggatcctggcaatttggaaaccgatctgcaagccggagccaaccacagatatgagctgctctgggtgattctgattggactcatcttcgcacttatcatacagtcgctagcagctaatcttggagtggttacagggaggcatctggctgagatctgcaagagtgagtaccccaagttcgtcaagattttcctatggctgctggcagagttggccgtcatcgctgcagatatcccagaagttatagggacggcctttgctttcaacatattgttccatattccggtgtgggtcggcgtcctcatcaccggcaccagcactctactgcttcttggcctccaaaaatacggggtgaggaagctggagtttctgatatcgatgctggtgttcgtgatggcggcgtgcttcttcggggagctgagcatcgtgaagccgccggcgaaggaggtgatgaaggggctcttcatccccaggctcaacggcgacggcgccaccgccgacgccattgccctcctcggagctcttgtcatgccccacaatctgttcttgcattctgccttggtgctatcgaggaagacaccggcatcagtcagaggaatcaaggacgggtgcaggttcttcctgtacgagagcgggttcgcgctgttcgtggcgctgctgataaacatcgccgtcgtctccgtctccggcaccgcctgctcctccgccaacctctcccaagaggacgccgacaagtgcgccaacctcagcctcgacacctcctccttccttctcaagaacgtgctgggcaagtcgagtgcgatcgtgtacggcgtggcactgttggcatctgggcagagctccactattaccggcacatacgctggacagtacatcatgcagggtttcttggacatcaggatgaggaagtggcttcggaacctgatgacaagaaccatcgccatcgcgccgagcctcatcgtctccatcatcggcggctccaggggcgccggccgcctcatcatcatcgcttcgatgatactgtccttcgagctgccgtttgctctcatccctcttctcaagttcagcagcagtaagagcaagatggggccccacaagaactctatctatataatagtgttctcgtggttcctgggtctgctcatcatcggcatcaacatgtacttcctgagcacgagcttcgtcggctggctcatccacaacgacctccccaagtacgccaacgtgctcgtcggcgccgccgtcttcccgttcatgctcgtctacatcgtcgccgtcgtctacctcaccatcaggaaggactccgtcgtcaccttcgtcgccgactcctccctcgccgccgtcgtcgacgccgagaaggccgacgccggcgacctcgccgtcgacgacgacgagcccttgccgtaccgcgacgacctggccgacatcccgctcccaaggtag

SEQ ID NO: 3 - DNA, 1635 bp, Oryza sativa
atggagattgagagagagagcagtgagagagggagcatcagctggagagctagtgcggcacatgatcaagatgccaagaagctcgacgcagatgatcagctgctaatgaaggagcctgcatggaaaaggttccttgcccatgttggtcctggattcatggtgtctttagcctacttggatcctggcaatttggaaaccgatctgcaagccggagccaaccacagatatgagctgctctgggtgattctgattggactcatcttcgcacttatcatacagtcgctagcagctaatcttggagtggttacagggaggcatctggctgagatctgcaagagtgagtaccccaagttcgtcaagattttcctatggctgctggcagagttggccgtcatcgctgcagatatcccagaagttatagggacggcctttgctttcaacatattgttccatattccggtgtgggtcggcgtcctcatcaccggcaccagcactctactgcttcttggcctccaaaaatacggggtgaggaagctggagtttctgatatcgatgctggtgttcgtgatggcggcgtgcttcttcggggagctgagcatcgtgaagccgccggcgaaggaggtgatgaaggggctcttcatccccaggctcaacggcgacggcgccaccgccgacgccattgccctcctcggagctcttgtcatgccccacaatctgttcttgcattctgccttggtgctatcgaggaagacaccggcatcagtcagaggaatcaaggacgggtgcaggttcttcctgtacgagagcgggttcgcgctgttcgtggcgctgctgataaacatcgccgtcgtctccgtctccggcaccgcctgctcctccgccaacctctcccaagaggacgccgacaagtgcgccaacctcagcctcgacacctcctccttccttctcaagaacgtgctgggcaagtcgagtgcgatcgtgtacggcgtggcactgttggcatctgggcagagctccactattaggccagtcacaatgggggtttcactggtgtgtcatgcacatttaatagggggtttcttggacatcaggatgaggaagtggcttcggaacctgatgacaagaaccatcgccatcgcgccgagcctcatcgtctccatcatcggcggctccaggggcgccggccgcctcatcatcatcgcttcgatgatactgtccttcgagctgccgtttgctctcatccctcttctcaagttcagcagcagtaagagcaagatggggccccacaagaactctatctatataatagtgttctcgtggttcctgggtctgctcatcatcggcatcaacatgtacttcctgagcacgagcttcgtcggctggctcatccacaacgacctccccaagtacgccaacgtgctcgtcggcgccgccgtcttcccgttcatgctcgtctacatcgtcgccgtcgtctacctcaccatcaggaaggactccgtcgtcaccttcgtcgccgactcctccctcgccgccgtcgtcgacgccgagaaggccgacgccggcgacctcgccgtcgacgacgacgagcccttgccgtaccgcgacgacctggccgacatcccgctcccaaggtag

SEQ ID NO: 4 - DNA, 1616 bp, Oryza sativa
atggagattgagagagagagcagtgagagagggagcatcagctggagagctagtgcggcacatgatcaagatgccaagaagctcgacgcagatgatcagctgctaatgaaggagcctgcatggaaaaggttccttgcccatgttggtcctggattcatggtgtctttagcctacttggatcctggcaatttggaaaccgatctgcaagccggagccaaccacagatatgagctgctctgggtgattctgattggactcatcttcgcacttatcatacagtcgctagcagctaatcttggagtggttacagggaggcatctggctgagatctgcaagagtgagtaccccaagttcgtcaagattttcctatggctgctggcagagttggccgtcatcgctgcagatatcccagaagttatagggacggcctttgctttcaacatattgttccatattccggtgtgggtcggcgtcctcatcaccggcaccagcactctactgcttcttggcctccaaaaatacggggtgaggaagctggagtttctgatatcgatgctggtgttcgtgatggcggcgtgcttcttcggggagctgagcatcgtgaagccgccggcgaaggaggtgatgaaggggctcttcatccccaggctcaacggcgacggcgccaccgccgacgccattgccctcctcggagctcttgtcatgccccacaatctgttcttgcattctgccttggtgctatcgaggaagacaccggcatcagtcagaggaatcaaggacgggtgcaggttcttcctgtacgagagcgggttcgcgctgttcgtggcgctgctgataaacatcgccgtcgtctccgtctccggcaccgcctgctcctccgccaacctctcccaagaggacgccgacaagtgcgcaacctcagcctcgacacctcctccttccttctcaagaacgtgctgggcaagtcgagtgcgatcgtgtacggcgtggcactgttggcatctgggcagagctccactattaccggcacatacgctggacagtacatcatgcagggtttcttggacatcaggatgaggaagtggcttcggaacctgatgacaagaaccatcgccatcgcgccgagcctcatcgtctccatcatcggcggctccaggggcgccggccgcctcatcatcatcgcttcgatgatactgtccttcgagctgccgtttgctctcatccctcttctcaagttcagcagcagtaagagcaagatggggccccacaagaactctatctatataatagtgttctcgtggttcctgggtctgctcatcatcggcatcaacatgtacttcctgagcacgagcttcgtcggctggctcatccacaacgacctccccaagtacgccaacgtgctcgtcggcgccgccgtcttcccgttcatgctcgtctacatcgtcgccgtcgtctacctcaccatcaggaaggactccgtcgtcaccttcgtcgccgactcctccctcgccgccgtcgtcgacgccgagaaggccgacgccggcgacctcgccgtcgacgacgacgagcccttgccgtaccgcgacgacctggccgacatcccgctcccaaggtag

SEQ ID NO: 5 - protein, 544 aa, Oryza sativa
MEIERESSERGSISWRASAAHDQDAKKLDADDQLLMKEPAWKRFLAHVGPGFMVSLAYLDPGNLETDLQAGANHRYELLWVILIGLIFALIIQSLAANLGVVTGRHLAEICKSEYPKFVKIFLWLLAELAVIAADIPEVIGTAFAFNILFHIPVWVGVLITGTSTLLLLGLQKYGVRKLEFLISMLVFVMAACFFGELSIVKPPAKEVMKGLFIPRLNGDGATADAIALLGALVMPHNLFLHSALVLSRKTPASVRGIKDGCRFFLYESGFALFVALLINIAVVSVSGTACSSANLSQEDADKCANLSLDTSSFLLKNVLGKSSAIVYGVALLASGQSSTIRPVTMGVSLVCHAHLIGGFLDIRMRKWLRNLMTRTIAIAPSLIVSIIGGSRGAGRLIIIASMILSFELPFALIPLLKFSSSKSKMGPHKNSIYIIVFSWFLGLLIIGINMYFLSTSFVGWLIHNDLPKYANVLVGAAVFPFMLVYIVAVVYLTIRKDSVVTFVADSSLAAVVDAEKADAGDLAVDDDEPLPYRDDLADIPLPR

SEQ ID NO: 6 - protein, 358 aa, Oryza sativa
MEIERESSERGSISWRASAAHDQDAKKLDADDQLLMKEPAWKRFLAHVGPGFMVSLAYLDPGNLETDLQAGANHRYELLWVILIGLIFALIIQSLAANLGVVTGRHLAEICKSEYPKFVKIFLWLLAELAVIAADIPEVIGTAFAFNILFHIPVWVGVLITGTSTLLLLGLQKYGVRKLEFLISMLVFVMAACFFGELSIVKPPAKEVMKGLFIPRLNGDGATADAIALLGALVMPHNLFLHSALVLSRKTPASVRGIKDGCRFFLYESGFALFVALLINIAVVSVSGTACSSANLSQEDADKCATSASTPPPSFSRTCWASRVRSCTAWHCWHLGRAPLLPAHTLDSTSCRVSWTSG

SEQ ID NO: 7 - DNA, 24 bp, Artificial
  primer Os7g2572_F2976g
tatattcagcctgggcagatcgag

SEQ ID NO: 8 - DNA, 24 bp, Artificial
  primer Os7g2572_R3815g
tgatgtactgtccagcgtatgtgc

SEQ ID NO: 9 - DNA, 29 bp, Artificial
  primer CNPorf5
caccatggagattgagagagagagcagtg

SEQ ID NO: 10 - DNA, 25 bp, Artificial
  primer CNPrt3
acacccttgtcgatcgatcgatctg

SEQ ID NO: 11 - DNA, 16 bp, Artificial
  primer M13 Forward
gtaaaacgacggccag

SEQ ID NO: 12 - DNA, 17 bp, Artificial
  primer M13 Reverse
caggaaacagctatgac

SEQ ID NO: 13 - DNA, 20 bp, Artificial
  primer CNP_GcheckFW
gcaagtcgagtgcgatcgtg

SEQ ID NO: 14 - DNA, 18 bp, Artificial
  primer CNP_GcheckRV
cgccgatgatggagacga